(19) United States
(12) Patent Application Publication
(10) Pub. No.: US 2026/0094698 A1
(43) Pub. Date: Apr. 2, 2026

(54) COMPUTERIZED METHOD AND SYSTEM FOR ASSESSMENT OF QUALITY OF LONG-TERM MANAGEMENT OF PATIENTS

(71) Applicant: B. G. NEGEV TECHNOLOGIES AND APPLICATIONS LTD., Beer-Sheva (IL)

(72) Inventors: Erez SHALOM, Gedera (IL); Yuval SHAHAR, Omer (IL)

(21) Appl. No.: 19/115,333

(22) PCT Filed: Oct. 22, 2023
(86) PCT No.: PCT/IL2023/051090

§ 371 Date: Mar. 26, 2025

Related U.S. Application Data
(60) Provisional application No. 63/418,556, filed on Oct. 23, 2022.

(51) Int. Cl. G16H 40/20

(52) U.S. Cl. CPC G16H 40/20

(57) ABSTRACT

Provided herein are computer implemented methods and systems for assessing long-term quality of management of patients in health care facilities. In particular, the methods and systems disclosed herein allow calculating guideline(s) adherence score, based on a calculated compliance score of a plurality of procedures of the guidelines(s), to thereby facilitate assessment of the quality of the long-term treatment of the patients.

### TECHNICAL FIELD

The present disclosure relates generally to computerized systems and methods for assessment of quality of long-term management of patients, based on compliance with corresponding management guidelines.

### BACKGROUND

An ageing population presents many challenges for health systems, including a shift towards care-based and end-of-life services, which leads to an increase in medical costs and long-term care. On the other hand, there is a growing shortage of professional care, leading to a heavy therapeutic burden on the care staff, as there are fewer physicians, interns and nurses in the nursing system. To improve medical care, many efforts are being made to base it on up-to-date evidence-based Clinical Guidelines (GLs).

The use of GLs may reduce the number of adverse events, such as misdiagnosis of a drug, or the occurrence of a preventable infection, and reduce the variability of care: It has been shown that GLs improve treatment uniformity, which may both increase the quality of physicians' decisions and increase patient survival rates while reducing morbidity, and may even reduce treatment costs.

However, despite all the benefits inherent in the use of GLs, its usage encounters a number of barriers: Most GLs are represented in free text and are usually inaccessible to the doctor at the point of care, especially when the doctor usually does not have the appropriate time and tools to search for the most appropriate GLs for the current patient. These reasons lead to low responsiveness and adherence to GLs on the part of physicians and low percentages of adoption among the medical staff.

To this aim, Clinical Decision Support System (CDSS) that assists care providers of various types in the automated application of GLs at the point of care has been developed. Such a system allows acquisition and formal representation of GLs in a formal manner comprehensible and interpretable by a computer, given the longitudinal digital patient record (Shalom E, et. al. Journal of Biomedical Informatics, 2016, 59:130-148).

Health Maintenance Organizations (HMOs) are constantly accrediting their hospitals, requiring them to comply with standards of quality and safety of care and the implementation of at least five GLs per year. Accurate and timely assessment of the quality of care and of the compliance to establish, evidence-based GLs, may support the effort to continuously enhance the quality of care and reduce its variance.

Thus, here is a need in the art for clinical decision support system(s) for assessing the quality of care, based on computerized clinical guidelines (GLs), in order to enhance medical care, reduce costs, save time, and overall enhance the capabilities of the clinical staff.

### SUMMARY

Aspects of the disclosure, according to some embodiments thereof, relate to advantageous computerized systems and methods for quality-assessment of long-term treatment of patients, under various settings, including, for example, geriatric patients at geriatric facilities, and the like. In some embodiments, based on the assessment of quality of care facilitated by the computerized systems and methods disclosed herein, enhancement of the medical care, enhancing capabilities of the clinical staff, reducing treatment costs and improving treatment time are achieved.

In some embodiments, there are provided herein, GL-based clinical decision support systems and methods, that can monitor retrospectively and accurately the level of performance of each GL and of GL components/procedures, even for crucial and complex GLs such as GLs for chronic diseases such as diabetes or blood pressure, or pressure ulcers GL for managing patients at geriatric hospitals, that can improve medical care management, reduce its variability, and expand the capabilities of the nursing staff and support the many tasks assigned thereto.

According to some embodiments, the computerized systems and methods disclosed herein, allows assessing the quality of care, by investigating the level of compliance of the clinical staff to clinical guidelines, over significant time periods, using retrospective longitudinal data. In some embodiments, the methods and systems disclosed herein make use of fuzzy temporal logic-based algorithm, which allows for determination of partial compliance of the clinical staff to quality metrics (for example, by assigning a score having a value of between 0 and 1 to each procedure (action), or predefined group of actions of a guideline), based on their value and on their temporal aspects. Thus, in some embodiments, temporal reasoning and fuzzy logic, are utilized to verify that the longitudinal application of a clinical guideline and to provide quantitative scores (for example, having a value of between 0 . . . 1) to the level of execution of each component of the guideline.

In some embodiments, the methods and systems disclosed herein are advantageous as they can provide a very detailed and accurate quality assessment at various levels of details (i.e., granularities of various stages, procedures and sub-components of a guideline), at various hierarchy levels (for example, of a single health care provider, a group of health care providers, a single shift, a specific ward, a specific health care facility, a specific group of patients, and the like).

According to some embodiments, there are provided herein a computerized method and system for quality control of medical care, based on the representation of existent medical guidelines. In some embodiments, the system may include a temporal fuzzy logic algorithm which considers for each action (procedure) its type, its weight within the overall treatment protocol, and its partial fuzzy logic membership function. According to some embodiments, there is further provided herein, a web-based business intelligence (BI) dashboard interface which can allow users to display the performance of care providers from different aspects, such as for various treatment stages, several hospital wards, different patient groups, etc.

According to some embodiments, the system enables the medical staff to, for example, identify gaps in the application of a particular treatment protocol, compare the quality assessment (QA) scores between different stages of the protocol (e.g., prevention versus treatment or follow-up stages), compare different clinicians, wards (departments) and even different time windows, thus enabling an early detection of patient-management problems.

In some embodiments, advantageously, using such quality assessment methods and systems regularly results in increased consistency and reduced variance in care (i.e., “noise”), in medical management of patients.

In additional embodiments, the methods and systems disclosed herein allow the health care providers (such as, paramedical staff, physicians) to closely monitor their own patients. For example, the system may be used to track protocol performance metrics in real time, and continuously, via the BI dashboard interface, and quickly, identify or aid in identifying patients for whom performance metrics are not adequate (for example, below a threshold). Such an immediate QA feedback can advantageously lead to an improvement in treatment.

According to some embodiments, the system may also assist in the transferring of therapeutic responsibility to the nurses, when performing protocol-based actions, thereby enhancing their empowerment, which, in addition to benefiting nurses, is also likely to free up time for physicians to address other, perhaps more clinically challenging problems. Furthermore, when a new protocol becomes available online, the methods and systems disclosed herein, has the ability, by providing direct and immediate feedback to the health care staff, after treatment and/or after a certain time period), to help the staff adjust the implementation of the protocol to the standard that is expected. Thus, the system can help preserve medical knowledge, apply it at the treatment point in a relatively short time, and distribute it among the staff.

According to some embodiments, as exemplified hereinbelow, the method and system can save time, whether used in automated or in semi-automated fashion. As the use of the system continues, and users become more experienced in operating the system for all its advanced functions, time savings will increase, while maintaining the same performance.

According to some embodiments, there is thus provided herein, a computerized method for assessing the quality of long-term management of patients at a health care facility, wherein the method includes the steps of: determining, on a selected type of management and a corresponding guideline, said guideline comprises a plurality of procedure, for each of the procedures in the guideline, a procedure compliance score, based on level of execution of the procedure and/or on temporal pattern of execution of the procedure; calculating an adherence score of the guideline, based on the plurality of the procedure compliance score(s); and assessing the quality of management, based on the guideline adherence score and/or one or more procedures compliance score.

According to some embodiments, the compliance score of the procedure may be determined/calculated based on the type and/or the characteristic of the procedure.

According to some embodiments, the procedure type or characteristic may be selected from binary procedure(s), temporal procedures, cyclical (periodic) procedure(s), or any combinations thereof. Each possibility is a separate embodiment.

According to some embodiments, the characteristics of the procedure may include binary constraint, cyclical (periodic) constraint, time constraint, entry-condition constraint, order constraint, multiple constraints, or any combinations thereof. Each possibility is a separate embodiment.

According to some embodiments, the procedure compliance score may be calculated based on binary calculation, proportional calculation, temporal fuzzy logic calculation, or any combinations thereof.

According to some embodiments, the method may include applying/utilizing temporal fuzzy logic algorithm(s) to obtain one or more of the procedure compliance score(s) and/or the guideline adherence score.

According to some embodiments, at least some of the procedures may be assigned a different weight for calculating the guideline adherence score.

According to some embodiments, at least some of the procedures may include one or more discrete components/actions/sub-actions.

According to some embodiments, at least some of the components of a procedure may be given a different weight, for the calculation of the compliance score of the procedure.

According to some embodiments, the guidelines may be comprised of sequential or parallel stages, each stage comprise one or more procedures. According to some embodiments, at least one of the stages may be given/assigned a different weight for the calculation of the guideline adherence score.

According to some embodiments, the guideline(s) may be predetermined, based on the type of management. In some embodiments, the guidelines are based on clinical databases/references and/or on expert knowledge. In some embodiments, the guideline may be computerized structured guideline, constructed using various computational tools, allowing the conversion of textual guidelines into computerized-accessible guidelines.

According to some embodiments, the method may further include displaying to a user one or more quality assessment related parameters. In some embodiments, the quality assessment related parameters may include, for example, but not limited to: guideline adherence score, one or more procedure compliance score(s), sub-scores of one or more components of a procedure, sub-scores of one or more stages of a guideline, or any combinations thereof. Each possibility is a separate embodiment.

According to some embodiments, the method may further include presenting/allowing a user to select presenting/allowing a user to browse one or more of, but not limited to: user one or more quality assessment related parameters of all patients, a selected group of one or more patients, one or more quality assessment related parameters over a selected time period, one or more quality assessment related parameters of selected health care providers, one or more quality assessment related parameters of selected wards of the health care facility, or any combinations thereof. Each possibility is a separate embodiment.

According to some embodiments, the method may further include presenting/allowing a user to select presenting one or more of, but not limited to: guideline adherence score of all patients, guideline adherence score of a selected group of one or more patients, guideline adherence score over selected time frames/periods, guideline adherence score of selected wards/departments of the health care facility, guideline adherence score of selected health care providers or any combination thereof. Each possibility is a separate embodiment.

According to some embodiments, the method may further include presenting/allowing a user to select presenting/allowing a user to browse one or more of, but not limited to: one or more procedure compliance score(s) of all patients, one or more procedure compliance score(s) of a selected group of one or more patients, one or more procedure compliance score(s) over selected time frame(s), one or more procedure compliance score(s) of selected health care providers, one or more procedure compliance score(s) of selected wards of the health care facility, or any combination thereof. Each possibility is a separate embodiment.

According to some embodiments, the heath care facility may be, for example, but not limited to: a hospital, a day care facility, a nursery home, a geriatric facility, and the like, or any combinations thereof. Each possibility is a separate embodiment.

According to some embodiments, the method is retrospective, i.e., quality assessment is retrospective.

According to some embodiments, the computerized method may include fuzzy logic algorithm(s). In some embodiments, the fuzzy logic algorithms may include a fuzzy temporal logic.

According to some embodiments, the procedure compliance score and/or the guideline adherence score may have a numeric score having any value in the range of, for example, between 0-1.

According to some embodiments, the method may be automatic. In some embodiments, the method may be semi-automatic. In some embodiments, one or more operating parameters of the method may be determined manually and/or automatically.

According to some embodiments, the method may further include providing a recommendation to a health care provider, based on one or more procedure compliance score(s) and/or the guideline adherence score.

According to some embodiments, there is provided a system for assessing quality of long term management of patients at a health care facility, the system includes at least a processor which is configured to execute a method which includes one or more of the steps of: determining for each of the procedures in a selected guideline (selected in accordance with the type of management), a procedure compliance score, based on level of execution of the procedure and/or on temporal pattern of execution of the procedure; calculating an adherence score of the guideline, based on the plurality of the procedure compliance score(s); and assessing the quality of management, based on the guideline adherence score.

According to some embodiments, there is provided a system for assessing quality of long-term management of patients at a health care facility, the system includes at least a processor which is configured to execute a method for assessing the quality of long-term management of patients as disclosed herein.

According to some embodiments, the system may include or be communicatively associated with one or more of: a display, a user interface, a memory, a localized server, a remote server, a data base, a knowledge database, or any combinations thereof. Each possibility is a separate embodiment.

According to some embodiments, there is provided a non-transitory computer-readable medium storing processor executable instructions on a computing device, when executed by a processor, the processor executable instructions causing the processor to perform the method for assessing quality of long-term management of patients, as disclosed herein.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not drawn to scale. Moreover, two different objects in the same figure may be drawn to different scales. In particular, the scale of some objects may be greatly exaggerated as compared to other objects in the same figure.

In block diagrams and flowcharts, optional elements/components and optional stages may be included within dashed boxes.

In the figures:

FIG. 1—a flowchart of steps in a method for assessing long-term quality of management (treatment) of patients, according to some embodiments;

FIG. 2—a schematic block diagram illustration of an exemplary system for assessing long-term quality of management of patients, according to some embodiments;

FIG. 3—an exemplary screen shot of the “Gesher” interface for defining/determining procedural knowledge for a guideline, according to some embodiments;

FIG. 4—a schematic illustration of an exemplary protocol (guideline) for the treatment of pressure ulcers in its textual form, as defined by the domain experts, according to some embodiments. The protocol has four main steps: (1) Admission, (2) follow-up, (3) follow-up and prevention, and (4) prevention, follow-up and treatment;

FIG. 5—an exemplary screen shot of pressure ulcer protocol formalized within the “Gesher” medical knowledge acquisition tool, according to some embodiments. The plan hierarchy is displayed on the bottom left; the declarative concepts are shown on the bottom right;

FIG. 6A—an exemplary illustration of a fuzzy logic trapezoid for determining a score, according to some embodiments. Point A defines the value below which the performance is not valid, and therefore the score will be 0. The interval of values from Point B to Point C defines the range of values for which the performance is complete. Any value between A and B is a partial performance, and as it gets closer to B the score will get closer to 1. Point C defines the value from which the performance is partial, and therefore the score is partial. Point D defines the value beyond which the performance is not valid, and a therefore the score of 0. Any value between C and D denotes a partial performance, and thus is assigned a partial score;

FIG. 6B—an exemplary illustration of a fuzzy logic trapezoid representing a time constraint of the action “the test should be performed within 72 hours from admission, and not later than 120 hours”. The trapezoid defines that a maximal score of 1 if the test is done within 72 hours, a score of 0 if the test is done after 120 hours and a partial score between 0 to 1 if test is done between 72 to 120 hours, using a linear function;

FIG. 6C—an exemplary illustration of a fuzzy logic trapezoid representing a cyclical constraint, in which a full score is assigned to the actions if the gap between instances is smaller than 72 hours, a partial score is received if the gap is between 72 and 80 hours, and a score of 0 is assigned if the gap is greater than 80 hours;

FIG. 7—an exemplary user interface for presenting procedure compliance scores and guideline adherence score; according to some embodiments;

FIG. 8A—line graph showing the time taken by a nurse to manually score all the quality metrics of each patient; and

FIG. 8B—line graph showing the time taken by the nurse to score all the quality metrics of each patient with the computerized system's support.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the following description, various aspects of the invention will be described. For the purpose of explanation, specific details are set forth in order to provide a thorough understanding of the invention. However, it will also be apparent to one skilled in the art that the invention may be practiced without specific details being presented herein. Furthermore, well-known features may be omitted or simplified in order not to obscure the invention.

According to some embodiments, there are provided herein computer implemented methods and systems, for allowing assessment of quality of long-term management/care/treatment of various types of patients under various management routines, in various types of health-care facilities.

According to some embodiments, the automated quality-assessment systems and methods disclosed herein can provide, for complex clinical care over significant time periods, quality-measure scores that can enable health care providers (such as a nurse, after a short training, to quickly and accurately assess the quality of care. In addition to its accuracy, the system considerably reduces the time taken to assess the various quality measures; time saving is expected to increase as users become more experienced operating the system. Decision-support systems may empower the nursing staff, enabling them to manage more patients, and in a more accurate and consistent fashion, while reducing costs

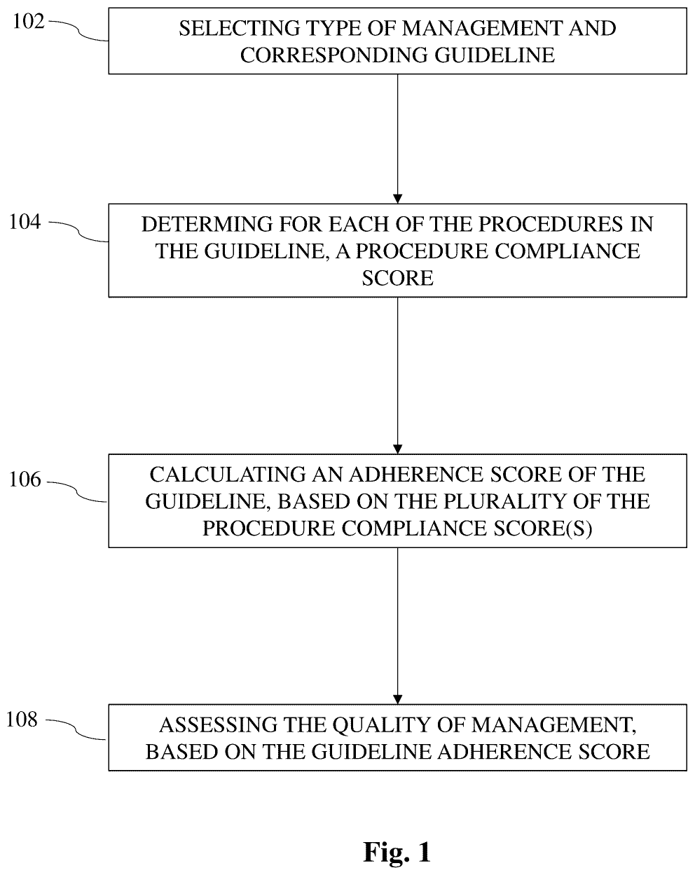

Reference is made to FIG. 1, which shows a flowchart of steps in a method for assessing quality of long-term management of patients. As shown in FIG. 1, at optional step 102, the type of management (for example, treatment) and the corresponding guideline(s) are selected. In some embodiments, the selection may be made by a user (for example, a health care provider). In some embodiments, the selection may be made automatically or semi automatically, for example, by the quality assessment system (as detailed below). The selection may be made from a predetermined database of guidelines (which may be created automatically, manually, or by expert supervision), as detailed herein below. In some embodiments, the selection of management may be made based on the characteristics of the patient(s), characteristics of the health care facility, characteristics of the ward at the health care facility, and the like. In some embodiments, the guidelines may include one or more procedures (for example, a plurality of procedures), that, in some cases/scenarios may be grouped in one or more stages. In some embodiments, one or more of the procedures may include components/sub-actions. Next, at step 104, for each of the procedures in the guideline, a procedure compliance score, is determined/calculated, based on level of execution of the procedure and/or on the temporal pattern of execution of the procedure, as further detailed herein below. In some embodiments, the procedure compliance score may have any value of between 0 (i.e., not executed at all) and 1 (i.e., fully executed as expected), thereby accurately representing the quality of execution of the procedure. In some embodiments, the compliance score may be calculated based on the on the type and/or characteristic of the procedure, as further detailed herein. Next, at step 106, based on the obtained/determined/calculated procedure compliance score(s), an adherence score of the guideline is calculated/determined. As detailed below, the calculation may take into consideration various characteristics, and may include, in some cases, assigning different weights to different procedure compliance score(s), to generate a guideline adherence score which represents as accurate as possible the quality of management. Next, at step 108, the quality of management is determined, based on the calculated guideline adherence score. In some embodiments, the quality of management is determined based on the calculated guideline adherence score and/or one or more of the procedure compliance score(s). In some embodiments, the quality of management is determined, at least partially based on the calculated guideline adherence score. In some embodiments, the quality of management is determined, at least partially based on the determined compliance score of one or more procedures. It is noted that as detailed hereinbelow, the guideline adherence score and/or one or more procedure compliance score(s) and/or any related parameters thereof, may be presented to a user at any stage of calculation, and, for example, at the selection of the user may represent the guideline compliance score of all or part of the monitored patients, during the entire time period, or portions thereof, thereby representing the corresponding quality assessment (i.e., for the entire cohort of patients, or only portions thereof, for the entire time period, or portions thereof, and the like). In some embodiments, as detailed below, the method may further include a step of providing a recommendation to a health care provider, based on the quality assessment, for example, with respect of the one or more procedures or sub procedures of the guideline.

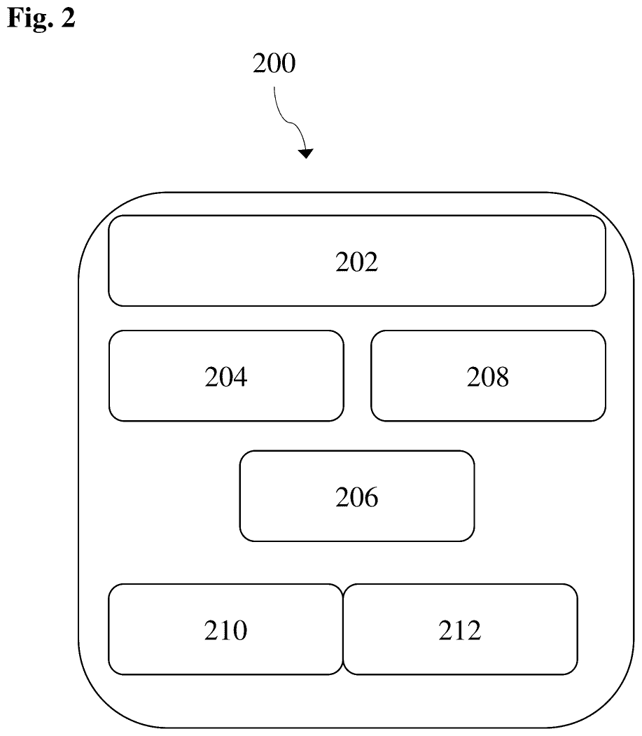

Reference is made to FIG. 2, which shows a schematic illustration of an exemplary system for assessing long-term quality of management, in accordance with some embodiments of the present invention.

According to some embodiments, system 200 includes a processor 202 configured to execute the method for assessing long-term quality of management of patients. According to some embodiments, the system 200 includes a memory module 204 in communication with the processor 202. According to some embodiments, the memory module includes a non-transitory computer-readable medium. According to some embodiments, the memory module includes stored commands. According to some embodiments, the commands stored onto the memory module 204 are configured to cause the processor 202 to execute the method as disclosed herein. According to some embodiments, the system 200 may include a user interface module 206. According to some embodiments, the user interface module 206 may be in communication with the processor 202. According to some embodiments, the user interface module 206 may include one or more of a monitor, a touch screen, a keyboard, a pointing device, a display, one or more buttons, and the like. According to some embodiments, the user interface module 206 may include or present the guidelines (at different detail levels). According to some embodiments, the user interface module 206 is configured to be operated/controlled by an operator/user. According to some embodiments, the system 200 may include and/or be in communication with a database 208 and/or knowledge base 210. According to some embodiments, the database 208 and/or knowledge base 210 may be stored within the system 200. According to some embodiments, the database 208 and/or knowledge base 210 may be stored onto a cloud. According to some embodiments, the processor 202 may be in communication with the database 208 and/or knowledge base 210. According to some embodiments, the database 208 may include data associated with one or more EMRs, healthcare facility related information, general clinical information, and the like. In some embodiments, knowledge base 210 may include data associated with one or more guidelines, procedures, outcomes, formal representation of the GLs, procedural knowledge, declarative knowledge, quality assessment knowledge, and the like, as further detailed below. In some embodiments, database 208 and knowledge base 210 may be comprised in a single module. In some embodiments, database 208 and knowledge base 210 are functionally and/or physically associated.

According to some embodiments, as used herein, the terms “guideline” and “GL” may be used interchangeably. The terms relate to a series of clinical or other types of instructions/procedures making a treatment/management scheme of one or more clinical conditions. In some embodiments, the series of procedures are ordered (sequence and/or timewise) and one or more of the procedures may have a temporal pattern. In some embodiments, the GLs may be divided into stages. The GLs may be determined by health care providers or may be determined automatically. The GLs may be predetermined or may adjusted.

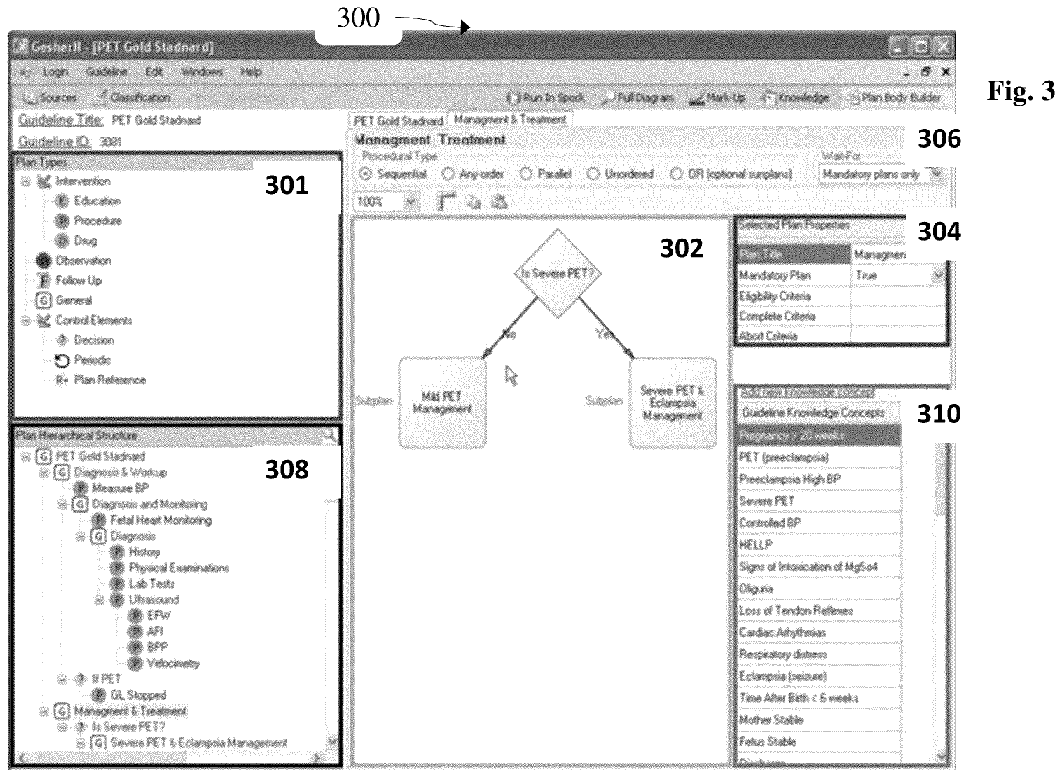

In some embodiments, in order to use textual GLs (i.e., GLs including a list of procedures), for the computerized methods and systems disclosed herein, the GLs may be converted from its textual representation to a formal, machine comprehensive representation. Such conversion may be performed, for example, using Digital Guideline Library (DegeL) architecture (Shahar Y, et. al., Journal of Biomedical Informatics, 2004; 37 (5), 325-344). DeGeL includes tools for converting GLs from its textual representation to a formal, machine comprehensive representation in the Asbru GL specification language (Hatsek A, et. al., Stud Health Technol Inform 2008; 139:203-12). In some embodiments, the conversion includes knowledge acquisition. In some embodiments, the conversion includes a gradual conversion process of the GL from its textual, unstructured (free-text) representation (which is usually done by medical experts), through its semi-structured representation (which is usually performed by knowledge experts and medical experts), to its formal, structured representation (usually performed by knowledge experts). This conversion may be performed using graphical tools, for acquiring knowledge, such as, for example, but not limited to: “Gesher” tool (Shalom E, et. al., Journal of Biomedical Informatics, 2008; 41 (6), 889-903). An exemplary screenshot of which is presented in FIG. 3. As shown in FIG. 3, the interface of the GESHER tool 300, assists in the performance of knowledge acquisition. Using Gesher tool, clinicians can define, with the help of knowledge engineers, the hierarchy of treatment plans, i.e., the procedural knowledge of the GL. Presented in FIG. 3, is part of the plan “Management and Treatment” of the preeclampsia-management GL. The overall plan starts by an assessment of the patient's condition, classifying it as either a severe or a moderate preeclampsia. The user selects management plans of different types (301) and adds them to the hierarchical flow chart (302). For each plan, several properties can be defined (304). For plans composed of several sub-plans, procedural knowledge attributes, such as the order of performance of the plans (parallel, serial, etc.) are also displayed (306). The sub-plan hierarchy is displayed as a Tree view (308). At this stage, the expert also defines a list of declarative knowledge concepts relevant to the GL, such as “Severe Hypertension”, which will be elaborated in the next stages of knowledge acquisition and can be selected for further use, as needed (310).

In some embodiments, the formal representation of the GLs may be saved/stored in the knowledge base. In some embodiments, the GLs knowledge may include, for example: procedural knowledge (“How to”), declarative knowledge (“what is”) and quality assessment knowledge.

In some embodiments, procedural knowledge (“How to”) may include, for example, but not limited to: which medications to administer, how much, and when, and under what conditions to start or stop, and from which plans is the GL composed, including sequential, parallel, and periodic plans. In some exemplary embodiments, the procedural knowledge of GLs may be represented using the Asbru GL-specification language (Shahar, Y et. al. 1998, Artificial Intelligence in Medicine, 14 (1-2), 29-51).

In some embodiments, the declarative knowledge (“What is”) may include, for example, but not limited to: what does “renal insufficiency” or “bone-marrow toxicity grade 2” or “deteriorating liver functions over the past year” mean, what is its precise definition, which logical and probabilistic components is it composed from (e.g., does “moderate anemia” on Monday and Thursday mean that the patient had a week of “moderate anemia”?). In some exemplary embodiments, declarative knowledge may be represented using the Temporal-Abstraction Knowledge (TAK) language, an extension of the Knowledge-Based Temporal Abstraction (KBTA) ontology (Shahar Y., Artificial Intelligence 90.1-2 (1997) 79-133) and the CAPSUL periodic temporal patterns language (Chakravarty, S., and Shahar, Y. (2000). Annals of Mathematics and Artificial Intelligence 30 (1-4)).

In some embodiments, the Quality-Assessment knowledge (“Why, or to achieve What”?) may include, for example, but not limited to: what is the intended patient outcome of each part of the GL or of the overall GL? What is the intention underlying a correct process, such as “reduce the blood pressure by using diuretics or beta-blockers”? How to score the quality of a monitoring process in which the health care provider visited the patient on average twice out of the intended three times per day?), and the like. In some exemplary embodiments, Quality-assessment knowledge may be represented using the TAK language with a fuzzy temporal logic extension, as further detailed herein.

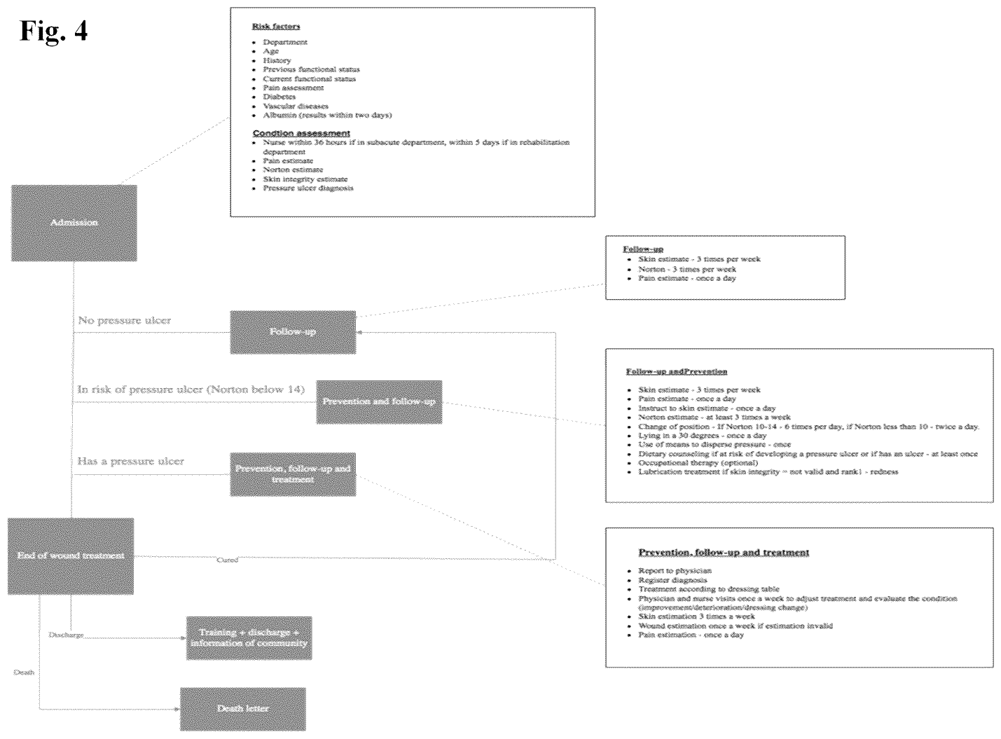

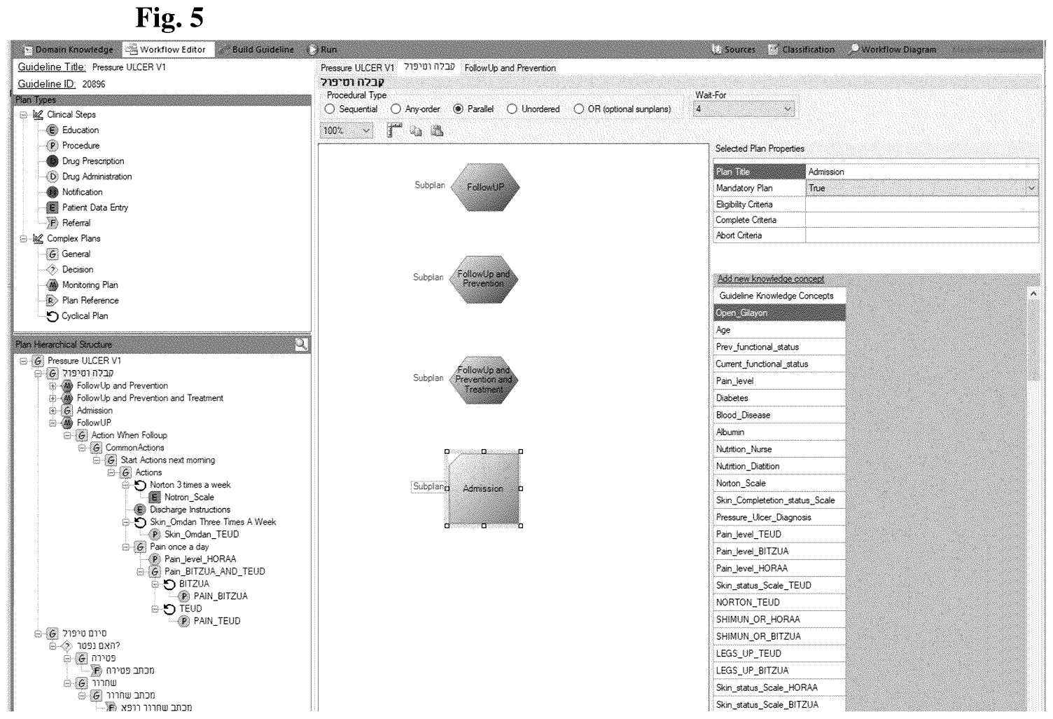

According to some embodiments, and for simplicity of description, pressure ulcer GL is used as herein an exemplary condition to be managed along with its corresponding guideline. However, it is clear that any other management scheme and associated guideline(s) can be utilized. The pressure ulcer GL is based on an existing clinical guideline for the prevention and treatment of pressure ulcers for hospitalized patients, and includes 4 main stages: (1) Admission, (2) follow-up, (3) follow-up and prevention, (4) follow-up, prevention and treatment. Each stage has a different pre-condition, and includes several procedures, such as bandages that should be applied, and different operations that need to be performed. The first step towards building a computerized GL, is to perform an Ontology Specific Consensus (OSC), as shown in FIG. 4. As detailed below, the OSC was validated with the clinical staff (health care providers) in order to ensure it represented the actual clinical practice. Then the GL may continue to be structured, leading to the final representation of the computerized GL. FIG. 5 presents an exemplary procedural knowledge of the GL modeled in the “Gesher” medical knowledge acquisition tool, and its various stages. The plan (GL) hierarchy is displayed on the bottom left; the declarative concepts are shown on the bottom right.

According to some embodiments, the guideline protocol includes, or is made up of a plurality of procedures/actions. Such actions may be ordered (sequential) and may be temporally distributed. In some embodiments, one or more of the procedures/actions may include or be made of one or more components/sub-actions. In some embodiments, each of the procedures may be characterized based on its type and/or various characteristics. In some embodiments, for one or more of the procedures, a compliance score may be determined/calculated, based on its type and/or characteristics, wherein the compliance score is determined based on the level of completion (execution) thereof and/or based on the temporal pattern of execution. In some embodiments, the compliance score of a procedure is determined based on the level of completion and on the temporal pattern of execution thereof.

In some embodiments, the types/characteristics of procedure may include the following constraints: Binary constraint, which includes actions that need to be performed only once, (e.g. performing the Norton test); Cyclical (periodic) constraint which includes actions that are performed in a pre-defined frequency (e.g., estimating pain once a day); Time constraint—which include actions that need to be performed within a certain period of time from a referenced event (for example, an albumin test that must be performed within two days after receiving the patient); Entry-condition constraint—which include actions that need to be performed when a specific entry condition is met, (for example, a patient at specific ward should receive nutritional advice from a dietitian); Order constraint which include actions that need to be performed in a specific order (i.e., before or after other action, for example, taking pain killer should be performed only after a pain assessment test.); Multiple constraints which include actions that need to be performed when at least one out of several conditions are met (e.g., given a symptom of redness in the heels, a silicone bandage or an olive oil bandage can be performed.); and Combination constraints which include a combination of constraints from any of the above. For example, the action “estimating pain once a day in the follow-up phase”, have a cyclical constraint (need to be performed once a day), an order constraint (need to be performed after an instruction to perform this action) and a binary constraint (the action needs to be indeed performed).

According to some embodiments, the constraint category (i.e., procedure characteristic/type) may be used to defines/determine the calculation type that is used to determine the compliance score. In some embodiments, the procedure (action) compliance score may be calculated by a binary calculation, a proportional calculation and/or a fuzzy logic algorithm.

In some embodiments, for a binary calculation, the compliance score is either 1 or 0, according to whether the action was performed or not (without penalty for unnecessary actions). Such calculation is suitable for procedures (actions) with a binary constraint, stating if the actions were performed or not. For example, the action “Norton test performed once at the admission stage will receive either 1, if performed, or 0, otherwise.

In some embodiments, for a proportional calculation the compliance score may be calculated according to the number of times (cardinality) that the action was performed, out of the total times that the action should have been performed. In general, the action's score is calculated according to the proportion of the time frame in which the action was actually performed, relative to the time frame required in the protocol. In some embodiments, such calculation may be used for actions with an entry-condition constraint—calculating, out of all the times when the entry condition has met, how many times the action was actually performed. For example, out of the times that the entry condition “pressure ulcer color is red with no slops or little slops” was met (the denominator), how many times the correct bandage was applied (the numerator). This is further exemplified in Example 2, below.

In some embodiments, a proportional calculation may also be used also actions with an order constraint—calculating out of all the times the action was performed, how many times it was performed in the correct order. For example, out of the times an instruction to perform a pain test was given (the denominator), how many times the paint test was performed (the numerator).

In some embodiments, a proportional calculation may also be used for actions with multiple-dependencies constraint—calculating out of all the times when at least one of the multiple entry conditions was met, how many times the action was performed. This is further exemplified in Example 3, below.

In some embodiments, a proportional calculation may also be used for actions with a cyclical constraint—calculating the performance rate in the expected time frame. In some embodiments, the score may be calculated using the formula:

$$\frac{cardinality\_Performed}{cardinality\_Expected * Time\_Proportion}$$

Where cardinality_performed is the number of times the action was actually performed, cardinality_expected is the number of times the action was expected to be performed, and time_proportion is the time interval between each instance, using a uniform distribution. For example, the action “perform a Norton test three times a week” has a time-window of one week, in this case, the interval between each instance (time_proportion) is expected to be 1.75. If four days have passed since the beginning of the week and a Norton test was performed only once, the score will be 1/(3*4/7)=0.5833

According to some embodiments, in the case of partial intervals observed, the cardinality expected is adjusted according to the proportion of the observed interval. For example, for an action expected to be performed 3 times a week, for an observed partial interval of 3 days since the proportion of the observed interval is 3/7, the cardinality expected will be adjusted from 3 to 3/7*3=1.28.

According to some embodiments, in case of two consecutive intervals, the gap between the last measurement in the first interval to the first measurement in the second interval may be measured.

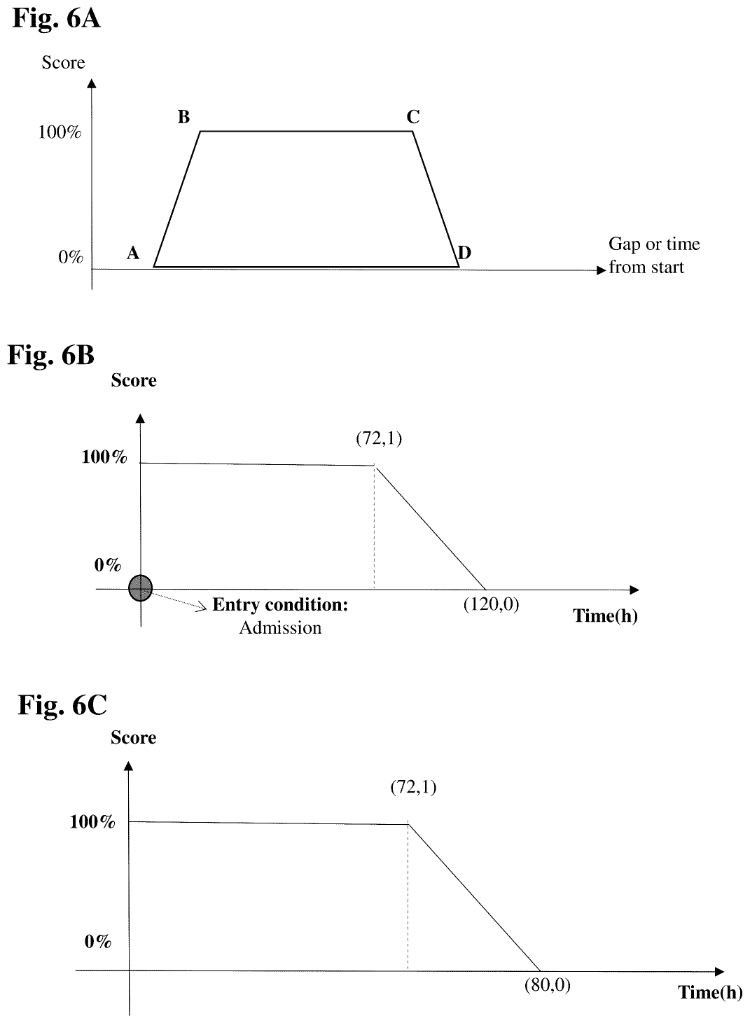

According to some embodiments, for the calculation of some compliance scores and/or adherence score(s), a (temporal) fuzzy logic function may be used. Such function/algorithm allows for a partial scoring of procedure compliance and/or adherence to the protocol's recommended action (i.e., guideline), using a fuzzy logic algorithm, based on a trapezoid schema: In some embodiments, each trapezoid is defined by up to 4 points. The score is determined by the trapezoid's points. An exemplary trapezoid is shown in FIG. 6A. The X-axis describes the time interval between an action and a subsequent action, and the Y-axis describes the score [0 . . . 100] given for that action given the interval. There are actions for which vertex A and B have the same value, and their X-axis value is even 0, i.e., no minimum interval is required between operations, and there are actions whose fuzzy-logic description requires all four vertices. As shown in FIG. 6A, the trapezoid maps the allowed time frame of an action to a score in the range [0-1]. Point A defines the value below which the performance is not valid, and therefore the score will be 0. The interval of values from Point B to Point C defines the range of values for which the performance is complete. Any value between A and B is a partial performance, and as it gets closer to B the score will get closer to 1. Point C defines the value from which the performance is partial, and therefore the score is partial. Point D defines the value beyond which the performance is not valid, and a therefore the score of 0. Any value between C and D (like any value between A and B) denotes a partial performance, and thus is assigned a partial score (i.e., having a value x, which is higher than 0 but lower than 1).

In some embodiments, a fuzzy-logic calculation may be used for actions with a time constraint—calculating the score according to the time defined in the trapezoid, from the beginning of the entry condition(s) until the action is performed. For example: for “perform an albumin test during reception within a day”, the protocol defines that if the test returns up to 72 hours from the admission, the score maximal, will be 1. If test is done within 72-120 hours a partial score will be assigned (using a linear function, between the points (72,1), (120,0). If test is done in more than 120 hours the score will be 0. The exemplary trapezoid in this case is shown in FIG. 6B. Shown in FIG. 6B, is the exemplary fuzzy-logic Trapezoid representing the time constraints of the action “the test should be performed within 72 hours from admission, and not later than 120 hours”. The trapezoid defines a maximal score of 1 if the test is done within 72 hours, a score of 0 if the test is done after 120 hours and a partial score between 0 to 1 if test is done between 72 to 120 hours, using a linear function.

Thus, for example, if the albumin test was performed after 80 hours from admission, the score will be 0.833:

$$m = \frac{0 - 1}{120 - 72} = - \frac{1}{48} , y = - \frac{1}{48} x + b$$

$$for ( x = 72 , y = 1 ) : b = \frac{1}{48} * 72 + 1 = \frac{5}{2}$$

$$Score == - \frac{1}{48} * 80 + 2.5 = 0.833$$

In some embodiments, a fuzzy-logic calculation may also be used for actions with a cyclical constraint—calculating the score according to the gap between each instance of the action. For example: for “perform a Norton test three times a week”, the protocol defines that the gap between the instances will be assigned a score of 1, if it is less than 72 hours, a partial score between 1 and 0 if the gap is between 72 and 80 hours, and a score of 0 if the gap is greater than 80 hours. Therefore, a test with a gap of 75 hours between the instances will receive, using the default linear function, a score of 0.625, as shown in the trapezoid displayed in FIG. 6C.

$$m = \frac{0 - 1}{80 - 72} = - \frac{1}{8} , b = \frac{1}{8} * 72 + 1 = 10 ,$$

$$score = - \frac{1}{8} * 75 + 10 = 0.625$$

According to some embodiments, based on the calculated compliance scores, an adherence guideline score may be determined/calculated. To this aim, in some embodiments, the various compliance scores may each be given similar or different weight. In some embodiments, the adherence score may be determined for the entire procedures, or to a portion of the procedures. In some embodiments, the adherence score may be determined for the entire patient cohort, or to a portion thereof. In some embodiments, the adherence score may be determined for the entire time period, or to portions thereof. In some embodiments, the adherence score may be calculated based on weighted average of at least some of the compliance scores.

According to some embodiments, in addition to the quality-assessment score (compliance score), each action/procedure may also have a weight representing its importance in the final calculation of the GL's adherence score, if needed. The final calculation of the GL's overall adherence score may thus be achieved by multiplying the procedure compliance score by the respective weight of the procedure, and summing the result over all actions. Thus, the systems and methods enable a knowledge engineer to also specify weights to each sub-action within a procedure or a stage, and also to each step within the protocol. This capability enables the knowledge engineer to penalize non-performance of actions that are considered to have greater importance, thus emphasizing the critical hospitalization stages.

In addition, the system and methods allow the knowledge engineer to assign weights to the action's different components. For example, different weights can be assigned to the explicit request (instruction) for performing an action, and to the actual performance of the action. Different weights can also be given for an action's performance frequency and to the correct order of its performance. The final score of the action may thus be calculated according to the weighted average of the action's components. An exemplary description of the weights of each step and action acquired from the domain experts is shown in Example 4.

Table 1 shows an example of the follow-up stage. Shown in Table 1 are exemplary weights acquired for the follow up-stage. In addition to the weight of the stage (22% in this example), weights are given also for each sub-action/procedure within the stage. For each action, weights can also be given to parts/components of the action (e.g., the temporal order (initiation or entry order) and the actual performance of the action), and also for different components of the score (e.g., frequency and orderEntry (Command provided).

TABLE 1

| | Stage | Sub-action/Procedure | | Parts/ | | Score | |
|---|---|---|---|---|---|---|---|
| Stage | weight | Component | Weight | Component | Weight | Component | Weight |
| Follow | 22% | Pain-once-a-day | 30% | Performance | 0.5 | Frequency | 0.5 |
| up | | | | | | Order (sequence) | 0.5 |
| | | | | OrderEntry | 0.5 | | |
| | | Skin-3-times-a-week | 35% | | | | |
| | | Norton-3-times-a-week | 35% | | | | |

According to some embodiments, after the various scores have been calculated, the results may be displayed to a user. To this aim, a dedicated interface may be utilized.

In some embodiments, a Business-Intelligence (BI) dashboard user interface that enables the medical staff/health care provider to view the scores of the actions in different protocol's stages may be used. In some embodiments, the scores (of one or more procedures, sub-procedures, stages and/or guideline), can be displayed for a selected group of patients, selected time frames, selected specific wards/departments, selected health care pr orders, and the like, at different levels of granularities (for example, a general score of the follow-up phase, and drill-down into the sub-scores of each action within the stage).

In some embodiments, the score of a specific time frame is the weighted average of the scores of all the sub-intervals within that time frame, taking into account the interval's sizes. The score of a time frame allows, for example, comparing between the medical staff's responsiveness during different time frames. For example, differences in the compliance during weekends compared to midweek, night shifts versus day shifts, etc.

In some embodiments, scores can also be calculated for a specific patient or group of patients. In some exemplary embodiments, the score of a group of patients is the average score of the patients within that group.

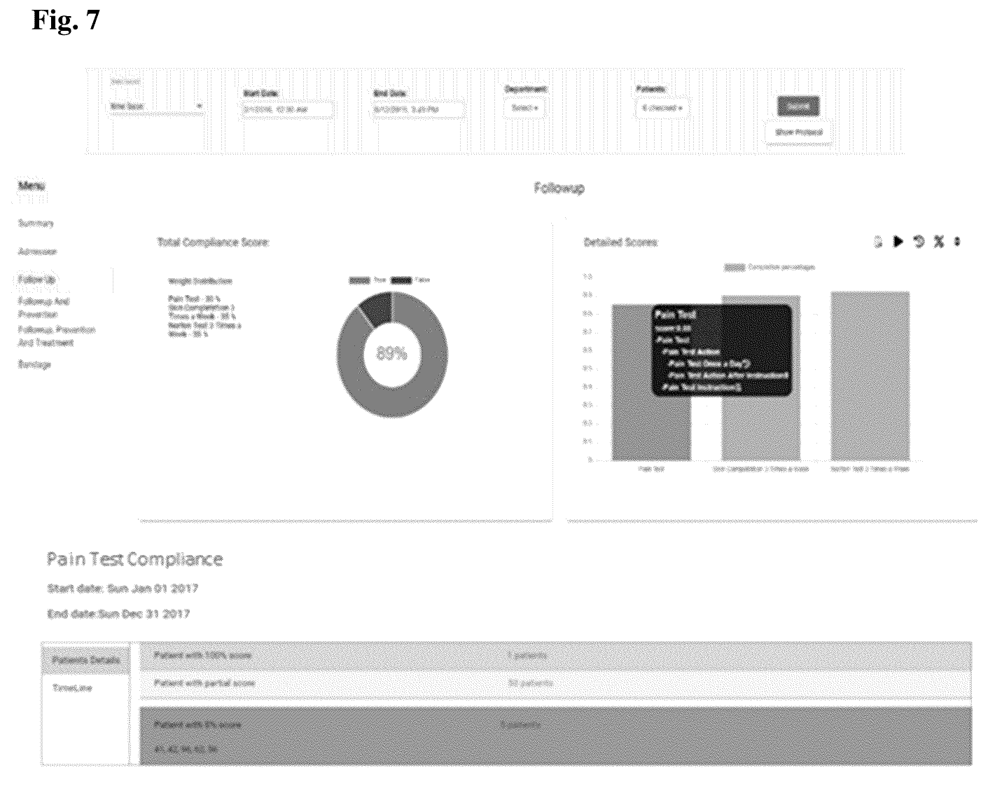

An exemplary user interface is presented in FIG. 7. As shown in FIG. 7, on the left hand, displayed are the different stages of the protocol: “admission”, “follow-up”, “follow-up and Prevention”, “FollowUp, Prevention and Treatment” and “bandage”. In the example shown in FIG. 7, the score of the current stage (follow-up), 89%, is calculated based on the weights and scores of each action this stage: (1) pain test, weighted 30% with a score of 85, (2) “skin completion 3 times a week” weighted 35% with a score of 90 and (2) “Norton test 3 times a week” weighted 35% with a score of 92. In the ruler at the top of the dashboard user interface, a specific time-frame to focus on may be selected, as well as specific departments/wards or group(s) of patients. At the bottom of FIG. 7 different scoring groups may be selected, for example: patients with 100% score, patients with 0% score and patients with a partial score. Each group contains all patients with the assigned score.

In some embodiments, the score may be a numerical value. In some embodiments, the score may be within a range of values. In some embodiments, for simplicity of calculation and representation the range of the score may be any numerical value of between 0-1, however, any other respective range of values may be utilized.

According to some embodiments, the heath care facility may be any type of facility, providing or capable of providing long-term or chronic treatment. Such facilities may include, for example, but not limited to: a hospital, a day care facility, a nursery home, a geriatric facility, and the like, or any combinations thereof. Each possibility is a separate embodiment. In some embodiments, the health care provider may be a physician, a nurse, an assistant, a paramedic, and the like.

In some embodiments, the management may include any type of management/treatment having a guideline. In some embodiments, the management may be of a chronic condition. In some embodiments, the management may be of a chronic patient condition. In some embodiments, the condition may include, for example, but not limited to: diabetes, hypertension, pressure ulcers, ulcers, dialysis, and the like.

In some exemplary embodiments, the management is of pressure ulcers (bed soars) of geriatric patients in geriatric wards (the health care facility). Bed soars is a condition from which 30% of hospitalized patients suffer, and about 17% to 28% in prolonged hospitalization.

According to some embodiments, the patient may be any type of chronic or long-term treated patient. In some exemplary embodiments, the patient is a geriatric patient. In some embodiments, the patient is an inpatient. Inpatients have various risk factors that should be monitored and checked in an organized manner as part of one or more guidelines, to identify deterioration of the patient's condition, such as restriction of mobility, malnutrition, lack of control of the sphincters and cognitive impairment. In some exemplary embodiments, the patient is suffering from hypertension diabetes, heart failure, renal failure, and the like, or combinations thereof.

According to some embodiments, the systems and methods may further be utilized to provide recommendation to a health care provider, based on the assessed quality. According to some embodiments, the system and methods disclosed herein may be used to provide a recommendation based on the quality assessment, for example if a score of the GL, a procedure, a stage, a group of procedures, and the like, is below a designated threshold. In some embodiments the threshold may be predetermined, adjusted, and the like. In some embodiments, for example, based on the procedures scores and/or the guideline compliance score(s), recommendation may be provided, in order to enhance compliance of a specific procedure, set of procedures and/or a guideline, to, for example, a single health care provider, a group of health care providers, a ward/department, a health care facility, and the like.

According to some embodiments, the guideline-based recommendations provided by the methods and systems can be provided to any type of health care provider and even to the patients and their families. Such recommendations are indeed based on the insights divulged by the time-oriented QA process provided by the methods and systems regarding the performance of the guideline. Thus, for example, using the systems and methods disclosed herein (i.e., based on the GL's formal representation, the temporal mediator, the QA analysis module, and the patient data) it is possible to determine (for one or more procedures and/or the entire guideline): one or more of:
[1] what has not been performed according to the GL (or has not been sufficiently performed according to associated determined quality score), that should be and can still be performed;
[2] what has not been performed and can no longer be performed;
[3] what should have been performed and was indeed performed beyond some sufficient measure;
[4] what should not have been performed but was performed;
and accordingly recommend what still needs to be performed in the near and far future according to the time-oriented guideline.

The present invention may be a system, a method, and/or a computer program product. The computer program product may include a computer readable storage medium (or media) having computer readable program instructions thereon for causing a processor to carry out aspects of the present invention.

A computer program (also referred to as a program, software, software application, script or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A computer program can be stored in a portion of a file that holds other programs or data, in a single file dedicated to the program in question, or in multiple coordinated files (for example, files that store one or more modules, sub programs or portions of code). A computer program can be deployed to be executed on one computer or on multiple computers that are located at one site or distributed across multiple sites and interconnected by a communication network.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, for example, JavaScript, Smalltalk, C, C++, TypeScript, Python and R.

The computer readable program instructions may execute entirely on the user's computer, partly on the user's computer, as a stand-alone software package, partly on the user's computer and partly on a remote computer or entirely on the remote computer or server (such as, a cloud based). In the latter scenario, the remote computer (or cloud) may be connected to the user's computer through any type of network, including a local area network (LAN) or a wide area network (WAN), or the connection may be made to an external computer (for example, through the Internet using an Internet Service Provider) including wired or wireless connection (such as, for example, Wi-Fi, BT, mobile, and the like). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention. Moreover, a computer can be embedded in another device, for example, a mobile phone, a tablet, a personal digital assistant (PDA, or a portable storage device (for example, a USB flash drive). Devices suitable for storing computer program instructions and data include all forms of non-volatile memory, media and memory devices, including semiconductor memory devices, for example, EPROM, EEPROM, random access memories (RAMs), including SRAM, DRAM, embedded DRAM (eDRAM) and Hybrid Memory Cube (HMC), and flash memory devices; magnetic discs, for example, internal hard discs or removable discs; magneto optical discs; read-only memories (ROMs), including CD-ROM and DVD-ROM discs; solid state drives (SSDs); and cloud-based storage. The processor and the memory can be supplemented by, or incorporated in, special purpose logic circuitry.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The processes and logic flows described herein may be performed in whole or in part in a cloud computing environment. For example, some or all of a given disclosed process may be executed by a secure cloud-based system comprised of co-located and/or geographically distributed server systems. The term “cloud computing” is generally used to describe a computing model which enables on-demand access to a shared pool of computing resources, such as computer networks, servers, software applications, and services, and which allows for rapid provisioning and release of resources with minimal management effort or service provider interaction.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

In the description and claims of the application, the words “include” and “have”, and forms thereof, are not limited to members in a list with which the words may be associated.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

The term “exemplary” with respect of specific figures of embodiments, refers to “an example”.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although stages of methods according to some embodiments may be described in a specific sequence, methods of the disclosure may include some or all of the described stages carried out in a different order. A method of the disclosure may include a few of the stages described or all of the stages described. No particular stage in a disclosed method is to be considered an essential stage of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to case understanding of the specification and should not be construed as necessarily limiting.

### EXAMPLES

### Example 1—Evaluation of the Quality Assessment of Pressure Ulcers Treatment Guideline in a Geriatric Health Care Facility

The research was performed in a health care facility which includes six inpatient wards (about 40 beds in each ward) and a dialysis institute (about 68 beds), with an average hospital stay lasting between weeks and several months, a fact that increases the risk of pressure sores in inpatients. The pressure ulcer treatment is performed by nurses who need to remember the complex textual guideline, making the adoption and implementation of the guideline by the already overloaded nursing staff quite difficult. As a result, the guideline's compliance is affected, leading to a decrease in the quality of care.

### The Data

100 random longitudinal patient records were retrieved from the EMR in which the term “Pressure Ulcer” appeared anywhere in the problems list. Each record included longitudinal data of several months of patient hospitalization in one of the departments participating in the study. The data mainly included measurements related to pressure ulcer treatment such as: age, Norton scale estimate, degree of ulcer severity, color, size and depth of ulcer, secretion and odor, relevant background diseases, nutritional estimate, dietary advice, skin integrity, posture change, pressure-dispersing measures, type of dressing, pain estimate, MUST estimate and patient guidance.

### The Knowledge

During the formalization process, the declarative knowledge was acquired, which included more than 140 different knowledge concepts that have been mapped to the hospital's medical database. Table 2 below describes the distribution of knowledge concepts acquired, using the Temporal Abstraction Knowledge (TAK) language in the different treatment stages.

TABLE 1
Knowledge concepts acquired

| Stage | Number of TAK files |
|---|---|
| Admission | 12 |
| Follow-up | 7 |
| Follow-up and prevention | 43 |
| Follow-up, prevention and treatment | 26 |
| Bandages | 52 |
| Total | 140 |

### The Technical Evaluation

In the technical evaluation, using retrospective patient longitudinal data, the feasibility of the system for quality assessment of the acquired GL was tested.

The technical evaluation was performed in two steps: In the first step, the algorithm, assessing whether the resulting output was as expected was evaluated. In the second step, a list of pre-defined quality-assessment patterns (such as “performance of the action before the instruction for it was given”) were evaluated by assessing their accuracy. For this purpose, data that were sufficiently realistic as to have a structure similar to that of the medical records retrieved, was simulated, and a visualization tool that displays in the data time-dependent templates for individual and multiple patients (“VISITORS” (Klimov D. et. al, 2010, Artificial intelligence in medicine, 49(1), 11-31)) was used to assess the correctness of each pattern, regarding time and value.

In both steps, all of the technical tests of the computational system were successful.

The BI interface's usability was evaluated by the senior nurse, using the standard usability scale (SUS) questionnaire (Brooke, J., et al. Usability evaluation in industry, 1996, 189.194:4-7), with a score of 90, indicating a high level of usability.

### Functional Evaluation

For the functional evaluation, 29 random patients and 30 quality metrics of care, relevant to the GL, were selected with the help of domain experts. A highly experienced senior geriatric nurse who was not part of the research team calculated the quality assessment scores of the treatment for all metrics, for each patient. Half of the patients were assessed by the nurse using the BI interface (i.e., using the computerized assessment method), after a short training with the system, and half of the patients were assessed manually, without the interface, using only the raw patient data.

The scores assigned manually by the nurse to each quality metric of care, were compared to the score given by the system, using a number of statistical tests such as: Spearman's non parametric correlation (to compare the ranking of the scores, for each metric, across all patients), and Pearson's Correlation (to compare the linear ordering of score values).

Also, the average time taken by nurse to perform the evaluation without and with the system for each group of patients measured and compared.

### The Functional Evaluation Results

### i) Comparison of Manual Quality Assessment (QA) to the Computerized (Automated) Quality Assessment System

In the manual evaluation, the senior geriatric nurse assigned scores manually to 15 out of the 29 randomly selected patients, for several quality metrics selected at random for each patient (a range of 9 to 17 metrics, with an average of 13 metrics per patient). Using an Excel spreadsheet as an electronic medical record, the nurse could relatively easily find the patient data needed to determine each quality metric's score.

The results, comparing the score assigned manually by the nurse to the score calculated by the automated system, are shown in Table 3 below. Note that a single patient can have several occurrences of the same quality metric, but at different stages of the protocol. For example, for the quality metric “reference to a dietician consultation” appearing 10 times in the protocol the spearman test comparing the ranks of the scores given by the system to the ranks of the scores given by the nurse had a result of R=0.853, p<0.01.

TABLE 2
Comparison between the quality metrics' scores assigned manually
by the nurse compared to the scores calculated by the automated system.

| | | Spearman test | | Pearson test | |
|---|---|---|---|---|---|
| Quality metric | #Appearances | R | p-value | P | p-value |
| Referral for dietary advice | 10 | 0.913 | 0.00022 | 0.981 | 0.0000005 |
| Nutrition (must) | 10 | same | | 0 | 1 |
| skin integrity estimation - | 11 | 1 | | 1 | |
| once a week | | | | | |
| Pain estimation - once a day | 14 | 0.596 | 0.02449 | 0.848 | 0.00012 |
| Norton estimation - | 15 | same | | 0 | 1 |
| admission | | | | | |
| Skin state estimation - | 15 | 0.559 | 0.03028 | 0.33 | 0.22966 |
| 3 times a week | | | | | |
| Skin state estimation - | 15 | 0.853 | 0.00005 | 0.853 | 0.000052 |
| on admission | | | | | |
| Change of position - | 15 | −0.034 | 0.90425 | −0.03 | 0.91547 |
| according to frequency | | | | | |
| Instruction Pain assessment - | 15 | same | | 0 | 1 |
| once a day | | | | | |
| Instruction for change of | 15 | −0.16 | 0.56894 | −0.155 | 0.581232 |
| position | | | | | |
| Document Norton - | 15 | 0.548 | 0.03444 | 0.834 | 0.000111 |
| 3 times a week | | | | | |
| Execution of dietary advice | 21 | 0.684 | 0.00062 | 0.734 | 0.0001521 |

It can be seen that most of quality metrics are significantly similar between the two vectors (nurse scores and system scores, for each metric, across all patients), some of them being simply identical.

Note in Table 3 that the correlation in the metric “change of position” (both the instruction and its performance) is not high. It turned out on further inquiry that the reason was because in rehabilitation wards, patients were allowed to get out of bed in the morning and in the evening; and since it did not matter when that was performed, the change of posture was simply not recorded in the medical record of the patient. It also turned out that this practice is “out of the protocol” and was done “unofficially” by some nurses in certain wards. It is worth noting that this informal practice was discovered by comparing between the scores of an automatic quality control system to the scores of a senior nurse.

Manual Quality Assessment Performance time

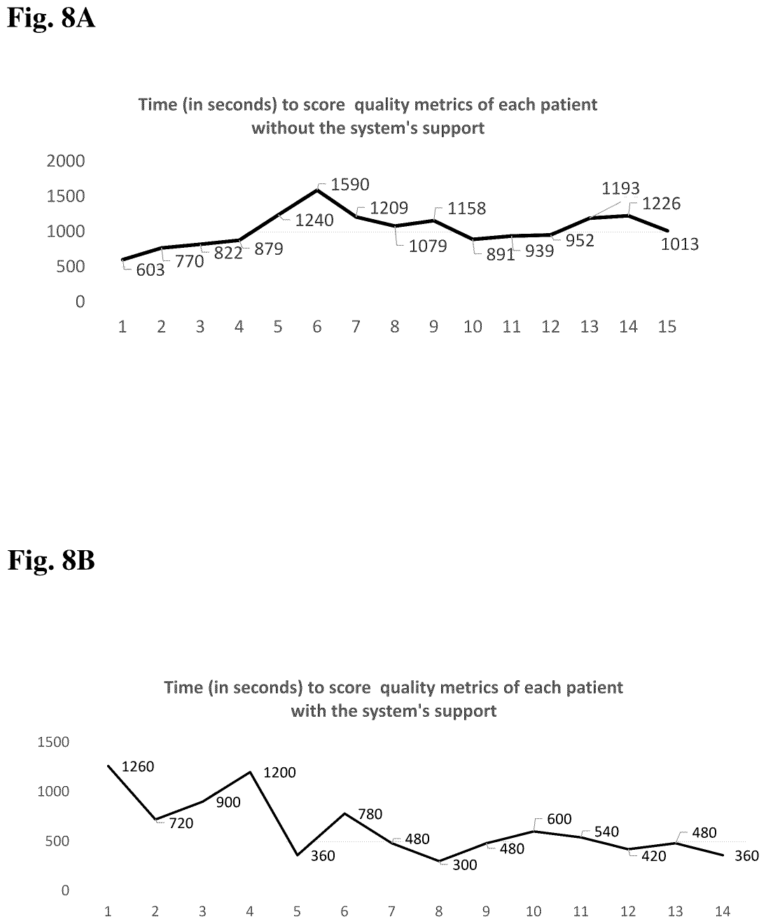

FIG. 8A shows a line graph of the time taken by the highly experienced senior nurse to manually calculate the scores of all metrics using the prepared spreadsheet for each patient.

As can be seen, the time for performing the quality assessment varies among patients, with an average of 1,039.29±242.4 seconds (i.e., a mean of approximately 17.3 minutes) to score on average 13 quality metrics per patient. Thus, scoring the QA scores for all of the 100 patients would be expected to require approximately 1730 minutes, or 28.8 hours, even for only the 13 metrics, on average, selected. It should be noted that using the hospital's EMR would be likely to have required an even longer time, due to the need to search for each relevant piece of raw data.

### Comparison of System-Supported Manual QA to Fully Automated System QA

Using the QA system's support, the same senior geriatric nurse using the BI (system) interface, assigned scores to the quality metrics to the other 14 patients out of the 29 randomly selected ones (a range of 9 to 15 metrics, with an average of 12 QA indicators per patient). For each metric, the nurse searched for its value, using the interface, and wrote separately its score for each specific patient.

The scores assigned by the nurse with the system's support were compared to the scores given by the automated system and retrieved by an experienced user of the system. Table 4 displays these results.

TABLE 3
Comparison of the quality metrics' scores assigned by the nurse when using the
system's support, to the scores assigned by the system and retrieved by an expert user.

| | | Spearman test | | Pearson test | |
|---|---|---|---|---|---|
| Quality metric | #Appearances | R | p-value | P | p-value |
| Norton estimation - admission | 14 | 1 | same | 1 | same |
| Skin state estimation - | 14 | −0.11289 | 0.70079 | −0.078566 | 0.78948 |
| 3 times a week | | | | | |
| Skin state estimation - | 14 | 1 | | 1 | |
| on admission | | | | | |
| Skin integrity estimation - | 12 | 1 | same | 1 | same |
| once a week | | | | | |
| Pain estimation - once a day | 13 | 0.72083 | 0.0054 | 0.89221 | 0.00004121 |
| Performance of correct | 13 | 0.88396 | 0.00006 | 0.87344 | 0.00009 |
| bandage for “red without or | | | | | |
| little secretion” | | | | | |
| Performance of dietary advice | 15 | 1 | same | 1 | same |
| Instruction Pain assessment - | 14 | 0.746 | 0.002151 | 0.700708 | 0.005247 |
| once a day | | | | | |
| Referral for dietary advice | 9 | 1 | same | 1 | same |
| Nutrition (must) | 8 | 1 | same | 1 | same |
| Norton Documentation - | 14 | −0.0769 | 0.7938 | −0.076923 | 0.793805 |
| 3 times a week | | | | | |

In terms of similarity in scores, it is noted that apart from the metric “skin state estimation—3 times a week” and “Norton documentation—3 times a week” all of the results were identical or highly significant, with most of the metrics having exactly the same scores. After investigating, it was found that the reason for the difference in both cases was the incorrect date range selection of the nurse in the interface, so that the score she saw was not the correct score. Another difference was that instead of a score of 98 given by the system, the nurse gave a score of 100, probably because she saw a very high indicator and did not pay attention to the exact value.

These results demonstrate that by using the system after a short training, a senior nurse is able to search and retrieve accurately the score for a desired quality indicator for each patient.

Manual Quality Assessment Performance Time when Assisted by the System

FIG. 8B shows a line graph of the time taken by the nurse to manually score all of the quality metrics for each patient. Note that there is a continuous improvement in the time taken to score all the quality metrics throughout patients. The mean time was 634.29±303.87 seconds to score approximately 12 quality metrics per patient on average.

It should be noted that, even in a largely automated system, there is a significant minimum time for manual assignment of quality scores, since for each metric, the nurse is required to perform a number of initial actions such as selecting dates, scrolling through the score tree to select another indicator, etc. FIG. 8B shows the learning curve of the tool, and the clearly decreasing trend in QA performance time.

Note also that already by the 14th patient, the nurse's speed in providing quality scores for all the QA indicators, with the support of the system, was three times faster, compared to her mean speed when not using the system (360 seconds, compared to a mean of 1226 seconds), as depicted in FIG. 8A.

### CONCLUSIONS

There were no significant differences (P<0.05) across the values of most quality measures given by the nurse manually compared to the values given by the system automatically. That is, in practice, at least in the pressure ulcers GL case, the system provides accurately and instantaneously quality assessment scores that are very similar to those of a highly experienced senior geriatric nurse.

There were also no significant differences (P<0.05) between the values of most quality measures when the nurse used the system manually to retrieve specific QA scores for specific patients, and the scores retrieved by an experienced knowledge engineer using the same system; i.e., by using the system after a very short training period, a senior nurse is able to find the correct values of the quality measure for each patient.

However, using the system's assistance significantly reduced the average assessment time it took for the nurse to score quality measures for each patient across all quality measures, reducing the time. By the 14th patient, the duration of the nurse's semi-manual assessment decreased to a third of her mean manual assessment time without the system's assistance.

### Example 2—Using a Proportional Calculation, for Actions with an Entry-Condition Constraint

In this example, it is calculated, out of all the times when the entry condition has met, how many times the action was actually performed.
Action: Dietary nutrition during the admission phase for all patients in the complex nursing department up to five days from the moment of admission.
Entry condition: Admission stage

Need to be checked that given that a patient is in the admission phase and at the complex nursing department—the patient received dietetic nutrition, and also that given that the patient received dietetic nutrition that he is indeed in the admission phase and in the complex nursing department.

The algorithm will perform the following exemplary steps:
1. Extract all the actions of dietary nutrition that have been carried out;
2. Extract all the actions of dietary nutrition that were performed when the patient was in the complex nursing department and during the admission phase;

Each patient will receive a performance score according to a trapezium function.
3. Select all patients in the complex nursing department and in the admission phase
4. Filter according to the requested time range
5. Calculate the number of operations that were performed when the patient was in the complex nursing department and in the admission stage out of the patients in the complex nursing department in admission
6. Count the number of operations performed when the patient was in the complex nursing department and in the admission phase out of all the dietary nutrition operations performed
7. optionally, weight the numbers calculated in step 4,5 with a weighted average

### Example 3—Using a Proportional Calculation for Actions with Multiple-Dependencies Constraint

In this example, it is calculated out of all the times when at least one of the multiple entry conditions was met, how many times the action was performed.
Action: Estimate pain once a day during the follow-up phase. Requires a pain-estimation command.

Here there are 3 types of calculations: cyclical (once a day), binary (command was given or not), and ordering (verifying that all performances were carried out after an instruction). For each calculation its score will be obtained, and then a weight is calculated according to the table of weights.

For example, for calculating the order score, the algorithm can perform the following steps:
1. Select all performances performed in the wrong order (after an instruction);
2. Extract all the intervals (according to the division of the periodic calculation, in this example, division into individual days);
3. For each interval check it contains an instance in the wrong order;
4. Filter intervals according to the requested time range;
5. Count how many intervals contain wrong actions from the total remaining intervals and return this score.

### Example 4—Description of Weights of Each Step and Action Acquired from the Domain Experts

The weights of various steps (stages) and procedures of the pressure ulcers guideline, which are used for determining quality compliance scores, are shown in Table 5 below:

TABLE 5

| | Stage | | | | | | |
|---|---|---|---|---|---|---|---|
| Stage | weight | Component | Weight | Component | Weight | Component | Weight |
| Admission | 26% | Albumin | 12% | | | | |
| | | Diet | 22% | Diet nurse | 0.5 | | |
| | | | | Diet nutritionist | 0.5 | | |
| | | Pain | 12% | | | | |
| | | Norton | 27% | | | | |
| | | Skin | 27% | | | | |
| Total | | 100% | | | | | |
| Follow-up | 22% | Pain once a day | 30% | Performance | 0.5 | Frequency | 0.5 |
| | | | | | | Order | 0.5 |
| | | | | Command | 0.5 | | |
| | | Skin 3 times a week | 35% | | | | |
| | | Norton 3 times a week | 35% | | | | |
| Total | | 100% | | | | | |
| Prevention and | 26% | Pain once a day (big | TBD | Performance | 0.5 | Frequency | 0.5 |
| follow-up | | estimation( | | | | Order | 0.5 |
| | | | | Command | 0.5 | | |
| | | Skin 3 times a week (big | 10% | | | | |
| | | estimation( | | | | | |
| | | Norton 3 times a week | 10% | | | | |
| | | (big estimation( | | | | | |
| | | lubrication (frequency | 10% | Performance | 0.5 | Frequency | 0.5 |
| | | according to table). | | | | Order | 0.5 |
| | | Olive oil, silicone, | | Command | 0.5 | | |
| | | baby cream | | | | | |
| | | Leg lifting, Norton | TBD | Performance | 0.5 | Frequency | 0.5 |
| | | below 14 | | | | Order | 0.5 |
| | | | | Command | 0.5 | | |
| | | Skin once a day | 10% | Performance | 0.5 | Frequency | 0.5 |
| | | | | | | Order | 0.5 |
| | | | | Command | 0.5 | | |
| | | Instruction | 5% | | | | |
| | | documentation once | | | | | |
| | | Change of position | 20% | Command | 0.5 | | |
| | | 6/9 times a day | | Performance | 0.5 | Frequency | 0.5 |
| | | | | | | Order | 0.5 |
| | | Lay at 10 degrees once | 15% | Command | 0.5 | | |
| | | a day | | Performance | 0.5 | Frequency | 0.5 |
| | | | | | | Order | 0.5 |
| | | Nutritionist | 10% | Nutritionist | 0.5 | | |
| | | consultation | | consultation | | | |
| | | | | Nurse | 0.5 | Reference | 0.5 |
| | | | | consultation | | Consultation | 0.5 |
| | | Pressure dissipation | 10% | | | | |
| Total | | 100% | | | | | |
| Prevention and | 21% | Pain once a day (big | TBD | Performance | 0.5 | Frequency | 0.5 |
| treatment | | estimation) | | | | Order | 0.5 |
| follow-up | | | | Command | 0.5 | | |
| (not including | | Skin 3 times a week (big | 15% | | | | |
| dressings) | | estimation) | | | | | |
| | | Norton 3 times a week | TBD | | | | |
| | | (big estimation) | | | | | |
| | | Skin once a day | 15% | Performance | 0.5 | Frequency | 0.5 |
| | | | | | | Order | 0.5 |
| | | | | Command | 0.5 | | |
| | | Change of position 6/9 | TBD | Command | 0.5 | | |
| | | times a day | | Performance | 0.5 | Frequency | 0.5 |
| | | | | | | Order | 0.5 |
| | | Lay at 10 degrees once | TBD | Command | 0.5 | | |
| | | a day | | Performance | 0.5 | Frequency | 0.5 |
| | | | | | | Order | 0.5 |
| | | Pressure dissipation | TBD | | | | |
| | | Leg lifting, Norton | TBD | Performance | 0.5 | | |
| | | below 14 | | Command | 0.5 | | |
| | | Report to doctor | 15% | | | | |
| | | Document diagnosis | 5% | | | | |
| | | Visit with doctor and | 15% | Signature | | | |
| | | nurse once a week | | Sore doctor visit | | | |
| | | | | Sore nurse visit | | | |
| | | Assess the integrity of | 15% | | | | |
| | | the skin once a week | | | | | |
| | | (wound assessment) | | | | | |
| Total | | 80% | | | | | |
| Dressings | 5% | Olive oil dressing | 34% | Frequency | 0.5 | | |
| | | | | Order | 0.5 | | |
| | | Foam and anti-odor | 33% | Frequency | 0.5 | | |
| | | dressings | | Order | 0.5 | | |
| | | Dressing Nacl0.9% | 33% | Frequency | 0.5 | | |
| | | | | Order | 0.5 | | |

1.-24. (canceled)

25. A computerized method for assessing quality of long-term management of patients at a health care facility, the method comprising:
selecting type of management and a corresponding guideline comprising a plurality of procedures;
determining, on a selected type of management and corresponding guideline comprising a plurality of procedures, for each of the procedures in the guideline, a procedure compliance score, based on level of execution of the procedure and/or on temporal pattern of execution of the procedure;
calculating an adherence score of the guideline, based on the plurality of the procedure compliance score(s); and
assessing quality of management, based on the guideline adherence score.

26. The method according to claim 25, wherein the procedure compliance score is calculated based on type and/or characteristic of the procedure.

27. The method according to claim 26, wherein the characteristics of a procedure comprise: binary constraint, cyclical (periodic) constraint, time constraint, entry-condition constraint, order constraint, multiple constraints, or any combinations thereof.

28. The method according to claim 25, wherein the procedure compliance score is calculated based on: binary calculation, proportional calculation, temporal fuzzy logic calculation, or any combinations thereof.

29. The method according to claim 25, wherein the method comprises applying temporal fuzzy logic algorithm(s) to obtain one or more of the procedure compliance score(s) and/or the guideline adherence score.

30. The method according to claim 25, wherein at least some of the procedures are assigned a different weight for calculating the guideline adherence score.

31. The method according to claim 25, wherein at least some of the procedures are comprised of one or more discrete components/actions.

32. The method according to claim 31, wherein at least some of the components of a procedure are given a different weight for the calculation of the compliance score of the procedure.

33. The method according to claim 25, wherein the guidelines are comprised of stages, each stage comprises one or more procedures.

34. The method according to claim 33, wherein at least one of the stages is assigned a different weight for the calculation of the guideline adherence score.

35. The method according to claim 25, wherein the guideline(s) are predetermined, based on the type of management.

36. The method according to claim 25, wherein the guideline(s) comprise computerized structured guideline.

37. The method according to claim 25, further comprising displaying to a user one or more quality assessment related parameters.

38. The method according to claim 37, comprising displaying to a user one or more quality assessment related parameters of all patients, a selected group of one or more patients, one or more quality assessment related parameters over a selected time period, one or more quality assessment related parameters of selected health care providers, one or more quality assessment related parameters of selected wards of the health care facility, or any combinations thereof.

39. The method according to claim 25, comprising allowing a user to select presenting to a user one or more of: guideline adherence score of all patients, guideline adherence score of a selected group of one or more patients, guideline adherence score over selected time frames, guideline adherence score of selected health care providers, guideline adherence score of selected wards of the health care facility, or any combination thereof.

40. The method according to claim 25, comprising allowing a user to select presenting to a user one or more of: one or more procedure compliance score(s) of all patients, one or more procedure compliance score(s) of a selected group of one or more patients, one or more procedure compliance score(s) over selected time frame(s), one or more procedure compliance score(s) of selected health care providers, one or more procedure compliance score(s) of selected wards of the health care facility, or any combination thereof.

41. The method according to claim 25, wherein the quality assessment is retrospective.

42. The method according to claim 25, further comprising providing a recommendation to a health care provider, based on one or more procedure compliance score(s) and/or the guideline adherence score.

43. A system for assessing quality of long-term management of patients at a health care facility, the system comprising a processor configured to execute the method of claim 25.

44. A non-transitory computer-readable medium storing processor executable instructions on a computing device, when executed by a processor, the processor executable instructions causing the processor to perform the method of claim 25.
